(19) United States
(12) Patent Application Publication
(10) Pub. No.: US 2016/0183038 A1
(43) Pub. Date: Jun. 23, 2016

(54) USE OF A WIRELESS DIRECT RADIOGRAPHIC PANEL IN MULTIPLE RADIOGRAPHIC EXPOSURE UNITS

(71) Applicant: AGFA HEALTHCARE, Mortsel (BE)

(72) Inventors: Wim GOVAERTS, Mortsel (BE); Geert LIEVENS, Mortsel (BE)

(21) Appl. No.: 14/910,160

(22) PCT Filed: Aug. 5, 2014
(86) PCT No.: PCT/EP2014/066832

§ 371 Date: Feb. 4, 2016

(30) Foreign Application Priority Data
Aug. 7, 2013 (EP) 13179512.2

(51) Int. Cl. H04W 4/00; H04W 12/04; A61B 6/00

(52) U.S. Cl. CPC H04W 4/008; A61B 6/4233; H04W 4/001; H04W 12/04

(57) ABSTRACT

A wireless direct radiographic panel for use in a medical imaging system including multiple radiographic exposure units each with different wireless settings, includes wireless settings adapted by moving the panel from one exposure unit to another. The adaptation of the wireless settings is occasioned by an information exchange occurring by moving an NFC tag installed on the panel within the operating range of an NFC tag installed on the workstation of the exposure unit wherein the wireless direct radiographic panel will be used.

### CROSS-REFERENCE TO RELATED APPLICATIONS

This application is a 371 National Stage Application of PCT/EP2014/066832, filed Aug. 5, 2014. This application claims the benefit of European Application No. 13179512.2, filed Aug. 7, 2013, which is also incorporated by reference herein in its entirety.

### BACKGROUND OF THE INVENTION

1. Field of the Invention

The present invention relates to a method for using a wireless direct radiographic panel in a medical imaging system comprising multiple radiographic exposure units, each with different wireless settings. The wireless direct radiographic panel is hereinafter often referred to as DR Panel.

More in particular the invention relates to a method and system for conveniently and operator-friendly swapping a wireless direct radiographic panel between the multiple radiographic exposure units of such medical imaging system.

A further advantage of the method and system according to the invention, is the convenient election of a wireless direct radiographic panel as active DR Panel for a radiographic exposure.

2. Description of the Related Art

It is known that radiographic illumination or exposure has important applications in medical imaging, whereby the medical advantages for the patient largely exceed the small risk of damage resulting from such radiographic illumination.

In earlier days radiographic exposures mostly made use of film based on silver halide technology as image capturing medium.

Since a number of years the so-called computed radiography technique has gained wide market acceptance. This technology makes use of a radiographic panel that does not use silver halide technology as the light capturing medium, but uses stimulable phosphors.

This method is described amongst others in detail in the Handbook of Medical Imaging, (ed. R. V. Matter et al., SPIE Press, Bellingham, 2000).

During recent years, radiographic exposures increasingly make use of direct digital radiographic techniques, known as DR (Direct Radiography).

This method is increasingly used as alternative for film-based imaging techniques, as well as for the panels based on the use of stimulable phosphor-technologies, as described supra.

In this digital radiographic method the radiographic exposure energy is captured pixelwise in a radiographically sensitive panel, and hereupon is converted to electronic image data by electronic components. Hereupon the information is read out imagewise and displayed on a suitable monitor for diagnostic purposes by a radiologist.

One of the driving forces behind the success of direct digital radiography is the ability to rapidly visualise the radiographic images and to efficiently and simply communicate over data networks to one or more sites for analysis and remote diagnosis by a radiologist or other medical expert. The delays that are characteristic for the development, packaging and physical transport of radiographic films are avoided by the above methods. Also the difficulties arising from the scanning of developed films and the corresponding loss in resolution is avoided by the above techniques.

The advantage of direct radiographic systems over computed radiographic systems, based on stimulable phosphors, is that no read-out (in a digitizer) of the latently captured radiographic image needs to take place. On the contrary, the digital radiographic image promptly or directly can be read for the purpose of evaluating the image from a diagnostic point of view. This diagnosis can take place at a local or remote workstation.

At the beginning the first direct radiographic panels were integrated in the overall radiographic imaging system. The wiring was designed such that minimal trouble to the radiographic operator was caused hereby when the radiographic direct panel was placed for exposure of a body part of a patient. More recently portable direct radiographic panels have been introduced to the market place. These panels make use of an on-board battery and communicate with the radiographic control panel or workstation, as well as with the data capturing apparatus and the display components in a wireless manner.

The latter aspects resulted in a wide acceptance of such portable wireless panels by the marketplace and ensures their practical use in a fully digital radiographic exposure system.

In a hospital or medical diagnosis center, these panels can be used as well in a completely newly installed radiographic imaging system or in a so-called retrofit situation. The term retrofit should be understood as directed to an existing radiographic system, that previously made use of radiographic films or stimulable phosphor plates, and whereby the latter registration media have been replaced by a direct radiographic capturing medium, a so-called direct radiographic or DR panel, without the need to replace the workstation or the radiographic source itself.

The advantage of such a retrofit radiographic system as compared to a completely newly installed radiographic system, is its lower investment cost, as part of the already installed radiographic system can be re-used.

Although portability and wireless communication of the radiographic registration medium clearly is an advantage when portable and wireless DR panels are used, these features also are characterized by the occurrence of problems under practical circumstances of use.

In particular problems occur when such DR Panels are used in a medical imaging system comprising multiple radiographic exposure units or stands.

A medical imaging system comprising multiple radiographic exposure units or stands may comprise for example on the one hand a mobile radiographic exposure unit, and on the other hand a fixed-position exposure unit. The latter exposure unit may further comprise for example a wall and/or a table stand.

One and the same wireless DR panel may as such be used in any of these exposure units. The wireless DR panel in such a configuration may ‘swap’ between any of these exposure units.

The problem however is when multiple radiographic exposure units are comprised in a medical imaging system, each of these units has its own radiographic workstation and access point, having its own settings for enabling wireless communication.

This implies that when the DR panel changes over or swaps from one radiographic exposure unit to another exposure unit, its wireless settings should be changed accordingly.

Differently phrased, its wireless settings should at any time match with the wireless settings of the radiographic work station and access point of the corresponding radiographic exposure unit wherein it is intended to be used.

When the wireless settings of the wireless DR panel have not been adapted accordingly, the communication between the DR panel and the controlling elements of the radiographic exposure unit wherein such DR panel is to be used, will give rise to mistakes. Such mistakes may occur for example when resetting the DR panel, or when transmitting the image and meta-data of the radiographic exposure to the work station of the radiographic exposure unit.

The above situation may be worsened when more than one wireless DR Panel is available in the various radiographic exposure units.

Contrary to radiographic films or stimulable phosphor panels that after exposure need to be removed from the radiographic exposure room for the purpose of being developed, resp. for being read-out in a digitizer, direct radiographic panels after use can remain in the radiographic exposure room.

When as a result of the above situation various direct radiographic panels are available in the radiographic exposure room, the radiographic operator needs to be fully sure that for the next or forthcoming radiographic exposure the right panel needs to be identified or selected.

Absent same it would be possible to address the wrong DR Panel, or to reset same, or the collect the data hereof.

Without a specific method that enables to reduce to an absolute minimum the probability of choosing a wrong DR Detector, there remains an enhanced risk for an incorrect exposure of a patient, resulting in retakes. On its turn, this results in a number of complaints, confusion, and a loss of time and efforts.

To cope with the above problems, Canon Inc., USA, has developed the following identification or selection method for direct radiographic panels, which it recommends for daily use. In the leaflet entitled ‘Canon CXDI-70C Wireless Premium Flat Panel Detector’, edited by Canon Medical Systems, A Division of Canon U.S.A. Inc., 15955 Alton Parkway, Irvine, Calif., USA, with reference DRB-014 Rev. A, 0611/2000, website www.usa.canon.com/csdi-70cwireless, a method for the identification/selection of digital radiographic panels has been described.(in the text that follows, both terms ‘identification’ or ‘selection’ of a direct radiographic panel is used, both terms having the same meaning)

The method described herein is as follows:

On the digital radiographic DR Canon Panels an infrared transmitter/sender is provided with pressure-sensitive button. This is the so-called IR check-in unit.

When a radiographic operator takes the Canon digital radiographic panel out of its docking station, he holds this panel on short distance before a radiographic workstation, wherein an infrared receiver is positioned.

Hereupon he pushes the IR pressure button, and the link to the radiographic workstation is unequivocally established.

As a result hereof the captioned direct radiographic panel is unambiguously and unmistakably identified and is ready for being used in the digital radiographic exposure unit.

During such identification the DR Panel receives the WIFI settings that are required to enable it to work in the setting of the radiographic workstation.

To this end, a fixed IP address, an SSID (Service Set Identifier) and a WPA-PSK (Wi-Fi Protected Access, Pre-Shared Key) of the access point and the IP address of the radiographic workstation is allocated to the DR Panel concerned.

The activation of the DR Panel as described above occurs in the Canon method by selecting the captioned panel on the workstation.

In US patent publication nr. US 2011/0116486 A1, published on May 19, 2011, in the name of Canon Kabushiki Kaisha, Tokyo, Japan, reference is made to the use of portable and wireless direct radiographic panels, and the identification of such panels by means of such infrared communication. For the purpose of identification of the DR Panel, ‘an input unit is provided for the X-ray sensor apparatus and accepts input from the user’. (Paragraph 33) Further in paragraph 35 is stated that ‘pressing the input unit of the X-ray sensor apparatus will start connection processing of the X-ray sensor apparatus to the access point.’ The term ‘pressing the input unit’ is repeatedly used in this specification, see e.g. par. 41, 4° line, par. 47, 5° Line, par. 51, 2° line, par 55 last but one line, etc.

Upon pressing the input unit, the wireless communication starts, based on the use of IrDA, Transferjet or UWB (paragraph 33).

In US Patent Application US 2010/0169423 A1, published Jul. 1, 2010, in the name of Konica Minolta Medical & Graphic, Inc, Tokyo, Japan, a radiographic image capturing systems is described, wherein a Flat Panel Detector (FPD) is activated and its IP address is communicated to the radiographic console, by means of pressing a pressure button by the radiographic operator. Reference is made to paragraph 58 stating that ‘ the operator depresses the button equipped on the FPD concerned . . . ’

### Problem to be solved

The method as described above with the Canon detectors gives rise to problems under practical use: the method is quite cumbersome, and hence it occurs that this procedure is not applied, in particular in emergency situations.

Same applies to the method described in the Konica & Minolta patent application. On top hereof, these methods may well be suitable when one DR panel is in use in a medical imaging system, but the method gives rise to problems when more DR panels simultaneously are available for use.

In a radiographic exposure room, in many cases various DR Detectors are used, as they differ for example in their respective sizes.

When a radiographic exposure is prepared from the central radiographic workstation or console, the correct panel should be selected for the forthcoming exposure. To this end the DR panel selected should not only comprise the wireless settings of the radiographic exposure unite wherein it shall be used, but also an unambiguous link is required between the radiographic exposure as planned and the corresponding DR Panel to be used.

As set forth supra, when the radiographic operator has selected the wrong DR Panel, the radiographic exposure cannot be effected correctly.

When the wrong panel is linked to the workstation, as soon as the exposure button has been activated, the wrong panel will start to integrate. This implies loss of image data.

The aim and purpose of the invention is to avoid the abovementioned problems by effecting an easy and unambiguous communication between the radiographic workstation of the radiographic exposure unit and the DR Panel as selected by the radiographic operator for the forthcoming radiographic exposure. By such a correct communication or information exchange, the DR Panel will be able to transmit its image and metadata to the workstation of the radiographic exposure unit, and also before the exposure, a wrongly selected DR Panel can be timely indicated at the workstation (this means, before the actual exposure takes place).

### SUMMARY OF THE INVENTION

The abovementioned aspects are realised by the method and the radiographic system as described below.

Specific features of preferred embodiments of the invention are set forth below.

Further advantages and embodiments of the present invention are clarified in the description that follows.

### DETAILED DESCRIPTION OF THE PREFERRED EMBODIMENTS

The present invention relates to a method and a radiographic system for using a wireless direct radiographic panel in a medical imaging system comprising multiple radiographic exposure units, each with different settings, wherein the wireless settings of the wireless direct radiographic panel are adapted upon moving the wireless direct radiographic panel from one exposure unit to another. More in particular such adaptation of the wireless settings according to the invention is occasioned by an information exchange occurring by moving an NFC tag installed on the wireless direct radiographic panel within the operating range of an NFC tag installed on the workstation of the exposure unit wherein the wireless direct radiographic panel will be used.

According to a preferred embodiment of the invention, the information exchange between both NFC tags occurs by an NFC tag installed on the radiographic workstation, pulling a response to occur from an NFC tag installed on the radiographic panel upon the latter being moved within the operating range of the NFC tag installed on the workstation.

According to a further preferred embodiment of the invention, as a result of the information exchange between both NFC tags and the corresponding adaption of the wireless settings on the wireless direct radiographic panel, wireless communication by short-range radio waves, preferably through WIFI, may occur between wireless communication processors installed on the direct radiographic panel and the workstation.

According to a further preferred embodiment of the invention, the adaption of the wireless settings of the wireless direct radiographic panel comprises communicating its unique identification code, e.g. its IP address, and adapting its private shared key, and its ESSID data.

According to a further preferred embodiment of the invention, as a result of the information exchange between the wireless direct radiographic panel and the radiographic workstation, the DR panel is retained as the active direct radiographic panel for the radiographic exposure in the radiographic exposure unit.

According to a further preferred embodiment of the invention, after selection of the direct radiographic panel as active panel and prior to the radiographic exposure, the conformity of the so selected direct radiographic panel with the direct radiographic panel set forth in the worklist of the radiographic work station for the radiographic exposure is checked.

Such worklist may be visualised on the radiographic workstation after navigating through the medical care organisation's or hospital's HIS or RIS system (HIS stands for Hospital Information System, RIS stands for Radiological Information System).

According to a further preferred embodiment of the invention, in case the conformity between the selected direct radiographic panel and the direct radiographic panel set forth in the worklist of the radiographic work station has not been established, a warning is given to the operator.

According to a further preferred embodiment of the invention, after selection of the direct radiographic panel as active panel and the occurrence of the radiographic exposure, the radiographic image and metadata stored in the direct radiographic panel are wirelessly transmitted to the radiographic workstation.

According to a further preferred embodiment of the invention, the direct radiographic panel selected as active panel is used for the radiographic exposures, until another direct radiographic panel has been selected as active panel.

Further, according to a preferred embodiment of the present invention, a medical imaging system is provided comprising multiple radiographic exposure units, each with different wireless settings, each such exposure unit comprising a radiographic workstation, the system comprising at least one wireless direct radiographic panel, an NFC tag being installed on each workstation and on the wireless direct radiographic panel, wherein the wireless settings of the wireless direct radiographic panel are adapted upon moving the wireless direct radiographic panel from one exposure unit to another, and wherein the adaptation of the wireless settings is occasioned by an information exchange occurring by moving the NFC tag installed on the wireless direct radiographic panel within the operating range of the NFC tag installed on the workstation of the exposure unit wherein the wireless direct radiographic panel will be used.

According to a preferred embodiment, the NFC tag installed on the direct radiographic panel is a passive NFC tag and the NFC tag installed on the workstation is an active NFC tag.

According to a further preferred embodiment, the workstation and the wireless direct radiographic panel further comprise wireless communication processors enabling the exchange of information by short-range radio waves, preferably through WIFI, the exchange of information being enabled by the adaption of the wireless settings of the wireless direct radiographic panel.

So, for the purpose of implementing the method and system of the present invention, the wireless direct radiographic panel and the workstation of the radiographic exposure unit both comprise an NFC tag installed thereon.

By moving the NFC tag installed on the direct radiographic panel within the operating range of the NFC tag installed on the workstation, an exchange of information will be occasioned. This exchange of information comprises the wireless settings of the radiographic exposure unit, so enabling the DR panel to wirelessly communicate with the various components comprised in the wireless network of the radiographic exposure unit. Such wireless communication occurs through a wireless communication processor installed on the DR panel.

Once the wireless settings of the radiographic exposure unit are received by the NFC tag installed on the DR panel, these are communicated to such wireless communication processor.

The term NFC tag as used in the present description should be understood as an electronic chip, designed specifically for being used according to the NFC set of standards. More in particular an NFC tag is essentially a small microchip containing a small amount of memory attached to an antenna/aerial which can store a small amount of information for transfer to another electronic device according to the NFC standard.

The information on the NFC tag is usually stored in a specific data format (NDEF-NFC data exchange format) so that it can be reliably read by electronic, e.g. mobile devices.

The NFC tags to be used in the method and system of the present invention preferably are no standard tags, as those won't work directly onto metal surface. To that end on-metal NFC tags should be selected, which are available e.g. from RapidNFC, London, U.K.

The term NFC as used in the present description should be understood as a set of standards for mobile devices such a smartphones and similar devices to establish radio communication with each other by touching them together or bringing them into close proximity, usually no more than a few inches.

NFC standards cover communication protocols and data exchange formats, and are based on existing radio-frequency identification (RFID) standards.

In a given medical imaging system comprising multiple radiographic exposure rooms, units or departments, a wireless digital radiographic detector can only be connected to a single radiographic workstation at the time.

When a DR panel connected to a specified radiographic exposure unit is taken by the radiographic operator for being used in another radiographic exposure unit, its wireless settings should be adapted accordingly.

Further, once the DR panel has received the appropriate wireless settings of the other exposure unit, according to a preferred embodiment of the present invention, before being used in such exposure unit, it should be selected as the active panel for such radiographic exposure, and—still more preferably—a prior control with the information included in the HIS/RIS/Worklist should take place.

### Link to the HIS/RIS/Worklist:

According to a preferred embodiment of the present invention, after identification or selection of the direct radiographic panel, the conformity of the so identified direct radiographic panel with the direct radiographic panel as set forth in the worklist of the radiographic work station for the forthcoming radiographic exposure is checked.

If the result of this conformity check is OK, the operator will proceed to the radiographic exposure. According to a still further preferred embodiment, in case the conformity between the so identified direct radiographic panel and the direct radiographic panel as set forth in the worklist of the radiographic work station has not been established, a warning is given to the operator. Such warning may comprise a pop-up on the display of the radiographic workstation, optionally including an acoustic or other form of alarm.

In such a case, a manual intervention of the operator is required: he can either adapt the worklist by selecting another DR Panel for the forthcoming exposure, for example the DR Panel identified as the active panel, or alternatively, he may select the DR Panel set forth in the worklist, and identify such panel as the active DR Panel.

The DR panel which was identified as the active DR Panel for an exposure, so remains the active panel for any further exposure, until another DR panel has been identified as the active DR panel.

According to a preferred embodiment, such newly identified DR panel shall only be accepted as the active DR Panel, after an positive conformity check with the radiographic worklist has been performed.

Once such new DR panel is indeed designated as the active DR Panel, it will on its turn keep such status, until another DR Panel is designated as the active DR Panel for forthcoming exposure(s).

The worklist of the planned radiographic exposures is usually displayed on the screen of the workstation during the various radiographic exposures that are planned for a given time-frame and for a given radiographic exposure room or unit.

Such worklist is part of or comprised within the Radiological Information System (RIS) of the hospital or medical care organisation and is communicated to the work station. Such communication may e.g. comprise the radiographic operator of the radiographic exposure unit concerned to navigate in the Hospital Information System (HIS) to the specific RIS data, and visualising on the screen or display of the radiographic work station such worklist. The radiographic worklist usually comprises one or more of the following information: identity of the patients to be radiographed (name or other personal attributes), object to be radiographed (arm, knee, hand, or other body part), stand (wall or bucky), as well as the digital radiographic panel to be used for the radiographic exposure, and—optionally—the exposure parameters.

### Unique Identification of DR Panel:

Each direct radiographic panel has a unique identification number or other form of identification. Such identification is allocated to the panel at the time of manufacturing the panel or at the time of marketing of the direct radiographic panel.

The abovementioned unique identification code or number of the direct radiographic panel may comprise or consist of a unique serial- or manufacturing-number, or, in an alternate embodiment, may comprise or consist of the fixed or variable IP address, MAC address or some sort of Unique Identifier allocated to the DR Panel.

The wireless communication module of the direct radiographic panel uses through the wireless communication protocol with the radiographic workstation this unique identification code to distinguish this DR Panel in an unambiguous manner from the other DR panels, and to identify same as such.

In a next step, namely after the radiographic exposure has taken place, the radiographic image data are sent to the radiographic workstation from the DR Panel that has been authenticated and registered to this end as the active DR Panel.

The authentication as registered DR Panel can only take place when at the time of instalment of the radiographic exposure room—or at the time of first use of the DR panel—the captioned DR Panel has been registered by its unique identification serial number or other form of identification by the radiographic workstation.

This is a typical administrative task that should not necessarily be performed by the radiologist, but can be taken care of by an administrative or technical assistant. Also the supplier or the approved or qualified installer of the radiographic exposure unit can take care hereof.

The latter then takes care of a (preferably wireless) data communication with the radiographic workstation, preferably by means of a WIFI or IEEE 802.11 network (a/b/g/n or the like).

The wireless communication module of the radiographic workstation, or the radiographic exposure unit, amongst others for the transmittal of the radiographic image data, is known for the person skilled in the art. Such module has been described e.g. in the US patents of Fuji Photo Film, Inc., Japan, Nr. U.S. Pat. No. 7,829,859 and U.S. Pat. No. 8,116,599. The patent first mentioned describes how the portable DR Panel transmits the digital image data stored in the DR panel over such wireless communication panel to the radiographic console by a transceiver of the DR Panel. The UWB (Ultra Wide Band) protocol is mentioned as an example of such wireless communication. Such UWB Protocol is characterised by a substantial reduction of energy-consumption, and by enhanced communication speed, as compared to other wireless communication techniques.

The other U.S. Pat. No. 8,116,599, describes the conversion to wireless communication signals of the image data by the wireless communication unit according to one of the following existing wireless communication protocols: UWB, Bluetooth, Zigbee, HiSWANa (High Speed Wireless Access Network type a), HiperLAN, Wireless 1394, Wireless USB, and finally Wireless LAN, infrared (irDA), NFC (Near Field Communication), IO-Homecontrol.

Preferably use is made of a wireless communication protocol working according to the IEEE 802.11 standard.

In such a case, the Direct Radiographic Panel communicates by a short-range radio or infrared connection over the wireless network with the radiographic workstation by of any of the above communication protocols.

Generally a short-range radio connection is preferred over an infrared connection, as the first mentioned connection operates in an omnidirectional manner, whereas for an infrared connection, as it is an optical connection method, a direct optical path should be created between the transmitter and the receiver of the signals.

The wireless LAN Network can make use of a number of various wireless network protocols and mechanisms. Preferably use is made of the wireless IEEE 802.11 g or IEEE 802.11 n interface (WIFI) standard.

One can also make use of the IEEE 802.11 b standard, whereby in a point-to-point configuration (1 point to various points), one access point (the wireless entry point) through a multidirectional antenna communicates with other clients that are within the range of the central access point.

The one access point is then the modality workstation, and the other clients are the various DR Panels, whereby one of these is identified/selected as the active panel.

So as to realise such wireless connection, preferably such WIFI connection, with the radiographic workstation, the processor has at its disposal on the direct radiographic panel an antenna driver and a chip technology that enables such short-range radio-connection.

In a radiographic exposure room the various direct radiographic panels mostly are placed in their respective docking stations. The docking station is the place where the direct radiographic panel is positioned when it is not used for a radiographic exposure: through such docking station the DR Panel recharges its on-board battery.

In the next step, once the radiographic exposure has taken place, the active DR panel will transmit its image data to the radiographic workstation, for visualisation and diagnostic evaluation on the monitor by a radiologist.

1-10. (canceled)

11. A method for using a wireless direct radiographic panel in a medical imaging system including a plurality of exposure units with different wireless settings, the method comprising the steps of:
adapting wireless settings of the wireless direct radiographic panel upon moving the wireless direct radiographic panel from one exposure unit of the plurality of exposure units to another exposure unit of the plurality of exposure units; wherein
the adapting of the wireless settings occurs in response to an information exchange due to moving an NFC tag installed on the wireless direct radiographic panel within an operating range of an NFC tag installed on a workstation of the exposure unit where the wireless direct radiographic panel will be used.

12. The method according to claim 11, wherein the information exchange occurs in response to the NFC tag installed on the workstation pulling a response from the NFC tag installed on the wireless direct radiographic panel upon the wireless radiographic panel being moved within the operating range of the NFC tag installed on the workstation.

13. The method according to claim 11, wherein, as a result of the information exchange and the adapting of the wireless settings on the wireless direct radiographic panel, wireless communication by short-range radio waves occurs between a wireless communication processor installed on the wireless direct radiographic panel and a wireless communication processor installed on the workstation.

14. The method according to claim 11, wherein the adapting of the wireless settings of the wireless direct radiographic panel includes communicating to the workstation a unique identification code, and adapting a private shared key and ESSID data.

15. The method according to claim 11, wherein, as a result of the information exchange, the wireless direct radiographic panel is retained as an active wireless direct radiographic panel for radiographic exposure in the exposure unit.

16. The method according to claim 15, wherein, after selection of the wireless direct radiographic panel as the active wireless direct radiographic panel, conformity of the selected wireless direct radiographic panel with a wireless direct radiographic panel in a work list of the workstation for the radiographic exposure is checked.

17. The method according to claim 16, wherein, in case the conformity between the selected wireless direct radiographic panel and the wireless direct radiographic panel in the work list of the workstation has not been established, a warning is given to an operator.

18. The method according to claim 15, wherein, after selection of the wireless direct radiographic panel as the active wireless direct radiographic panel and a radiographic exposure, a radiographic image and metadata stored in the wireless direct radiographic panel are wirelessly transmitted to the workstation.

19. A medical imaging system comprising:
a plurality of exposure units each having different wireless settings, each of the plurality of exposure units including a workstation with an NFC tag installed thereon; and
a wireless direct radiographic panel with an NFC tag installed thereon; wherein
wireless settings of the wireless direct radiographic panel are adapted in response to an information exchange due to moving the wireless direct radiographic panel from one of the plurality of exposure units to another of the plurality of exposure units; and
the information exchange occurs in response to the NFC tag installed on the wireless direct radiographic panel being within an operating range of the NFC tag installed on a workstation of the exposure unit where the wireless direct radiographic panel will be used.

20. The medical imaging system of claim 19, wherein the NFC tag installed on the wireless direct radiographic panel is a passive NFC tag and the NFC tag installed on the workstation is an active NFC tag.
